(19) United States
(12) Patent Application Publication
(10) Pub. No.: US 2019/0110988 A1
(43) Pub. Date: Apr. 18, 2019

(54) Delivery System That Utilizes Liposomal or Emulsion Vehicles

(71) Applicant: Lemulsa Technologies Inc., Dover, DE (US)

(72) Inventors: Tyler Barrett, Gulf Breeze, FL (US); Charles Sly, Plano, TX (US)

(73) Assignee: Lemulsa Technologies Inc., Dover, DE (US)

(21) Appl. No.: 15/786,032

(22) Filed: Oct. 17, 2017

(51) Int. Cl. A61K 9/127; A61K 47/46

(52) U.S. Cl. CPC A61K 9/1271; A61K 47/46

(57) ABSTRACT

The present invention discloses a liposomal delivery vehicle that includes lignite extract (or humic or fulvic acid) as a complexing agent to improve the stability and ultradeformability of the liposomal vehicle, thereby improving bioavailability. An emulsion delivery vehicle that includes lignite extract (or humic or fulvic acid) as the complexing agent is also disclosed. These improved delivery vehicles enhance the pharmacokinetic profiles of the active ingredients and reduce the quantities needs for therapeutic effect.

### TECHNICAL FIELD

This invention relates to a delivery system for pharmaceutical active ingredients, and more specifically to, the use of complexing agents within liposomal and emulsion vehicles to deliver active ingredients, such as sildenafil, to the patient.

### BACKGROUND OF THE INVENTION

Liposomal delivery vehicles have been widely researched and developed. Liposomes (lipid vesicles) are spherical bilayer vesicles, which are commonly formed from phospholipids, in which an aqueous solution is encapsulated. These vesicles are formed when thin lipid films or lipid cakes are hydrated and stacks of the bilayers become fluid and swell. These hydrated films detach during agitation and self-close to create multilamellar vesicles (LMV). Once these particles have formed, sonic energy (sonication) or mechanical energy (extrusion) is required to reduce the size of the particles. Properties of liposomal formations can vary depending on the composition, however, these same preparation methods are commonly used across all liposomes. The general steps to produce a liposome include (1) preparation of the lipid layers for hydration, (2) hydration with agitation, and (3) separation to a homogeneous distribution of vesicles.

Liposomes have become popular in recent years to deliver various active ingredients, homeopathic agents, traditional Chinese medical agents, nutraceuticals, and cosmeceuticals, due to their ability to protect these ingredients as they are delivered to the target location (i.e., cells, organs, bloodstream). The properties of the liposome are adjusted or revised to efficiently deliver the active ingredient to its target and to protect the active ingredient during that journey. For example, if the target location is an intracellular environment of an internal organ, then the membrane of the liposome should be robust, so that the aqueous solution inside can make it to the target. And if the target cells are difficult to enter or pass, then the membrane of the liposome may need to be ultradeformable, so that it is sufficiently flexible to make it to the target.

Various methods of producing liposomes and encapsulation of active ingredients or therapeutic agents have been disclosed. For example, U.S. Pat. Nos. 3,932,657, 4,311,712, and 5,013,556, and U.S. Patent Application Nos. 2012/0171280, 2016/0331693 disclose methods of preparing liposomes. These patents and patent applications are hereby incorporated by reference.

However, these methods fall short in efficiently delivering the active ingredients and other pharmaceuticals to their targets. Many of these methods fail to sufficiently protect the aqueous solution in the liposome from degradation on the journey to the target location and/or do not efficiently attach to the target cellular environment. Further, traditional liposomes may have an undesirable taste to the patient.

Many pharmaceutical companies have used emulsion vehicles to deliver various active ingredients, homeopathic agents, traditional Chinese medical agents, nutraceuticals, and cosmeceuticals to target environments in the human body. An emulsion vehicle is a fluid system in which liquid droplets are dispersed in a liquid, where the two or more liquids are normally immiscible (unmixable). In practice, an aqueous solution containing the active ingredient is mixed with a water solution to create the emulsion solution, where the liquid droplets are usually statistically distributed in the liquid matrix. These emulsion vehicles suffer from many of the same drawbacks as the liposomal delivery vehicles, and without the liposomal membrane to assist with protecting the active ingredient, traditional emulsions have difficulties delivering the active to the target location.

### BRIEF SUMMARY OF THE INVENTION

In the past, pharmaceutical companies were limited in terms of bioavailability because there was no way to push large particles, active ingredients, or cells through oral mucosal tissue or transdermally. Traditional solutions included using more active ingredients, decreasing the particle size of the active ingredient, using penetration enhancers, introducing the active ingredient via injection or infusion, or utilizing peglayed attachments to active ingredients. However, the use of lignite extract (or humic or fulvic acid) improves the stability and ultradeformability of the liposomal vehicle, thereby improving bioavailability for delivery vehicles with large particles, active ingredients, or cells, including stem cells. Pharmacokinetic profiles of the actives are also improved and the quantities needs for therapeutic effect are reduced. In alternative embodiments, lignite extract may be used (1) with humic acid and fulvic acid, (2) with humic acid or fulvic acid, or (3) by itself as a complexing agent for the liposome.

In some embodiments, the present invention utilizes an emulsion vehicle that contains a lignite extract (or humic or fulvic acid), which acts as a complexing agent for various active ingredients. This emulsion vehicle improves the bioavailability and the pharmacokinetic profiles of the active ingredients, while decreasing the quantity that needs to be taken for therapeutic effect. In alternative embodiments, lignite extract may be used (1) with humic acid and fulvic acid, (2) with humic acid or fulvic acid, or (3) by itself as a complexing agent for the emulsion vehicle.

Prior methods to improve poor active ingredient stability also focused on peglaytion of the active ingredient and pairing of the active ingredient with other substances that would prevent enzymatic metabolism. The use of lignite extract (or humic acid or fulvic acid) enhances the stability of the active ingredient and improves the ability to deliver the active ingredients to the target environments.

Traditionally, pharmaceutical companies have tried to combat poor/bitter taste with different types of flavoring or reducing particle size of the active ingredient. However, these features did not affect the active ingredient's ability to bind to g-protein coupled receptors (GPCRs) that are responsible for bitter taste sensation. The present invention uses lignite extract (or humic acid or fulvic acid) to bind to the GPCRs, which may assist with preventing the poor/bitter taste problems with prior delivery vehicles.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 shows a method for creating a liposome according to some embodiments of the present invention;

FIG. 2 shows the method for creating a liposome according to some embodiments of the present invention;

FIG. 3 shows the method for creating a liposome according to some embodiments of the present invention;

FIG. 4 shows a membrane of a liposome according to some embodiments of the present invention;

FIG. 5 shows a method for creating a liposome according to some embodiments of the present invention;

FIG. 6 shows the method for creating a liposome according to some embodiments of the present invention; and

FIG. 7 shows a method for creating an emulsion according to some embodiments of the present invention.

### DETAILED DESCRIPTION OF THE INVENTION

The present invention utilizes a liposomal vehicle that contains a lignite extract, which is similar to humic or fulvic acid, which acts within its aqueous phase as complexing agents for various substances such as sildenafil, pharmaceutical active ingredients, homeopathic agents, traditional Chinese medical agents, nutraceuticals, and cosmeceuticals to improve the bioavailability of these substances. This novel delivery system improves the pharmacokinetic profiles and decreases the quantity of these substances that need to be taken for therapeutic effect. This delivery system can be utilized for mucosal or transdermal delivery and is capable of delivering its payload to the intracellular environment. The lignite extract helps to deliver the payload intracellularly despite any early breakdown of the liposomal vehicle before it reaches the target cell because the lignite is capable of carrying the active ingredients into the intracellular environment independent of the liposomal vehicle. In alternative embodiments, lignite extract may be used (1) with humic acid and fulvic acid, (2) with humic acid or fulvic acid, or (3) by itself as a complexing agent for the liposome.

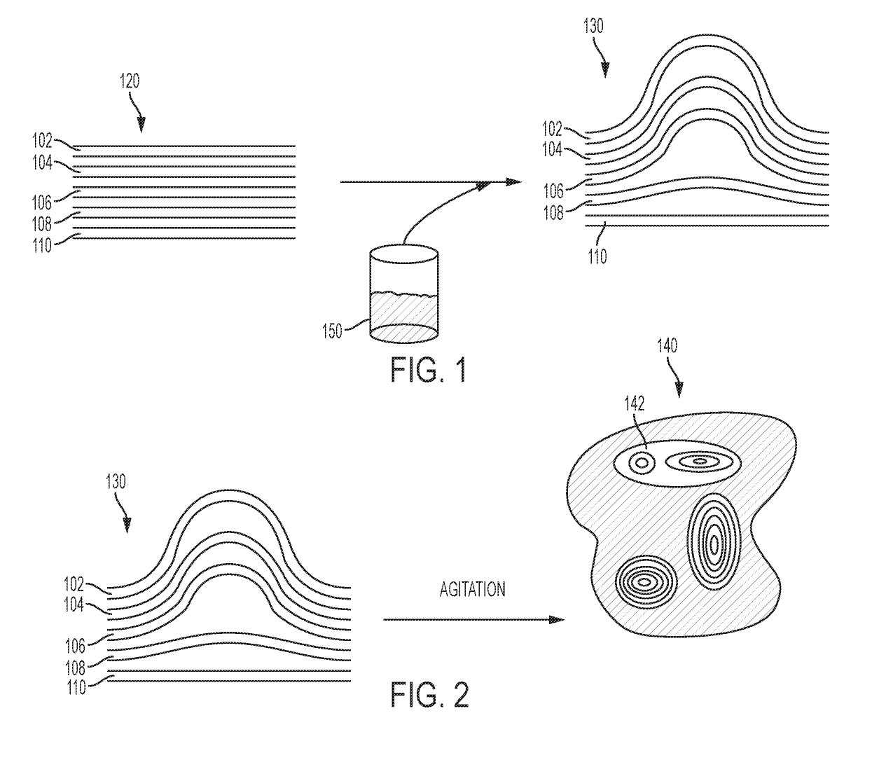

FIGS. 1 and 2 illustrate how liposomes are made in some embodiments of the present invention. A lipid film or lipid cake 120 comprises a first lipid layer 102, a second lipid layer 104, a third lipid layer 106, a fourth lipid layer 108, and a fifth lipid layer 110. A liquid solution 150, which normally includes the active ingredients and the complexing agents, is added to the lipid film 120, which causes swelling of the lipid film 130. As can be seen in the swelled lipid film 130, the first lipid layer 102 separates from the second lipid layer 104, which separates form the third lipid layer 106. The liquid solution 150 fills in the area between the lipid layers 102, 104, 106, 108, 110. Then agitation and potentially further hydration begin to detach and self-close the hydrated lipid film 130 into a solution 140 of large multiamellar vesicles 142. These large vesicles 142 may contain numerous lipid layers 102, 104, 106, 108, 110.

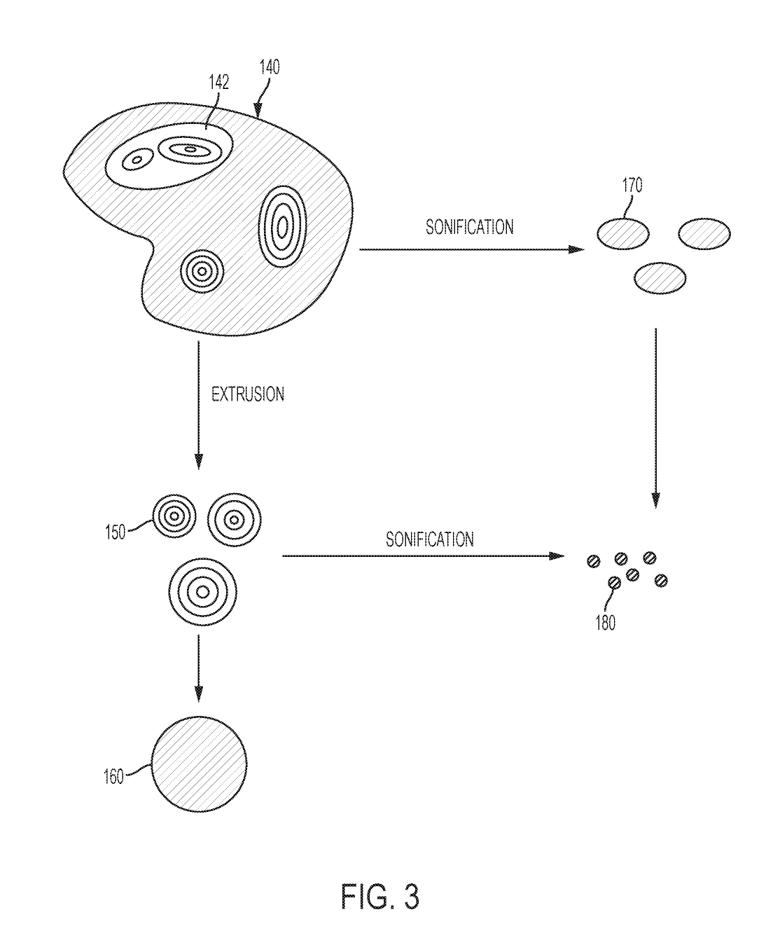

As illustrated in FIG. 3, in some embodiments these large multiamellar vesicles 142 must be reduced in size for practical applications. Sonification and extrusion methods are the most popular methods for reducing the size of these vesicles. As described below, extrusion can be used to create large unimellilar vesicles 150, which depending on their size may include one or more lipid layers 102, 104, 106, 108, 110. Reference numeral 150 shows a cross-section of the large unimellar vesicles, while reference numeral 160 shows the spherical shape of these vesicles. Sonification, which is further described below, may then be used to create small unimellar vesicles 180. Sonification may be directly used on the large multiamellar vesicles 142 to create disc-shaped small unimellar vesicles 170. Then the disc-shaped vesicles 170 are homogenized in the solution to create the small unimellar vesicles 180.

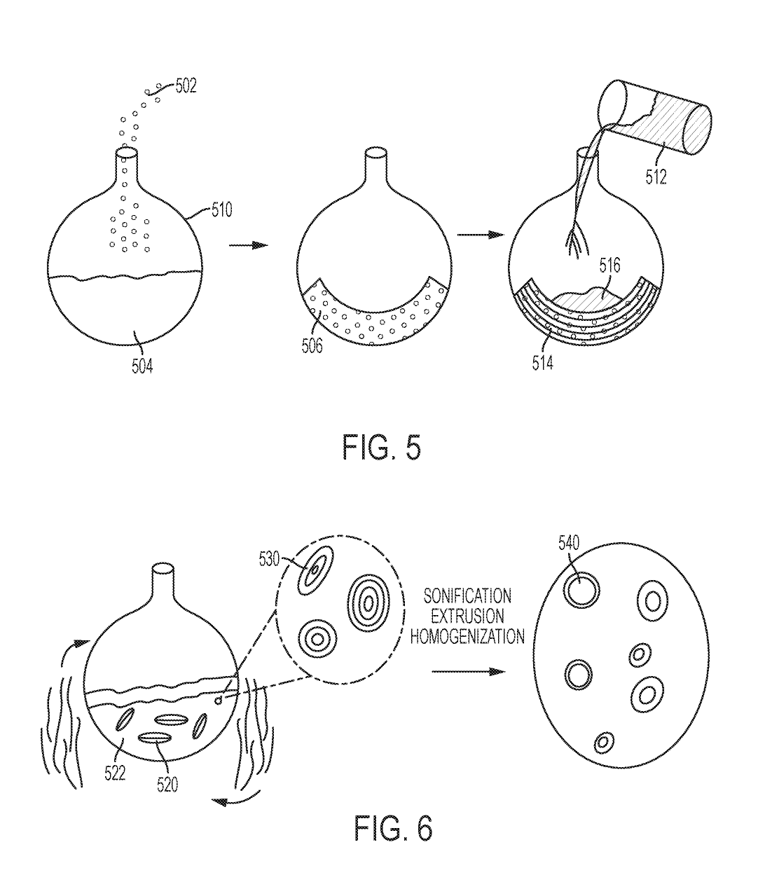

FIGS. 5 and 6 further illustrate the process of making a liposomal vehicle according to certain embodiments of the present invention. Starting with FIG. 5, a desired mixture of lipids (phospholipids) 502 are added to an organic solvent 504 and dissolved to create a homogeneous mixture of lipids (phospholipids). A beaker or container 510 may be used to hold the organic solvent 504 and homogeneous mixture of lipids (phospholipids). Chloroform or chloroform methanol may be used to create the homogeneous mixture. Once the lipids are dissolved and evenly distributed, the solvent is removed (evaporated) to yield a lipid film 506 in the container 510. The lipid film (cake) 506 is subsequently dried to remove any trace of the organic solvent 504. This drying process can also be done by freezing the lipid film and then placing the frozen lipid film on a vacuum pump and lyophilized until dry.

Then an aqueous solution 512 is added to the container 510 of dry lipid 506. The aqueous solution 512 should comprise the active ingredients, complexing agents, etc. that are desired in the liposome. As the aqueous solution 512 is added it interacts and hydrates the dry lipid film (cake) 506. Reference numeral 516 shows the aqueous solution 512 interacting with the lipid film (cake) 506 and reference numeral 514 shows the dry lipid film (cake) 506 hydrating and expanding. Now in FIG. 6, the hydrated lipid film (cake) 514 begins to separate as the solution is agitated. Thus, the solution 522 now contains the aqueous solution 512 and the separated hydrated lipid films 520. Continued agitation further separates the hydrated lipid films 520 into smaller and smaller multilamellar vesicles 530. These multilamellar vesicles 530 are found throughout the solution 522. Then, sonification, extrusion, and homogenization are used to create the unilamellar vesicles 540 from the multilamellar vesicles 530. The sizes of these unilamellar vesicles 540 are determined by the properties of the materials and substances involved, the methods of separation, the time and power used in separation, and the temperature during separation. For example, sonification generally produces smaller unilamellar vesicles (SUV), while extrusion produces large unilamellar vesicles (LUV). This information and additional information on the preparation of liposomes can be found at www.avantilipids.com.

Sonification is a process where sonic energy (sonication) is used on LMV suspensions to produce SUVs. The most common instruments to prepare sonicated particles are bath and probe tip sonicators. Cup-horn sonicators may also be used. Extrusion is a process where a LMV suspension is forced through a polycarbonate filter with a defined pore size to produce large unilamellar vesicles (LUV) that are close to the same pore size. Thus, the liposomes created from extrusion are generally larger than the liposomes generally created from sonification. As mentioned above, sonification may be used after extrusion to further reduce the size of the liposomes. Various methods of making liposomes are also disclosed in U.S. Patent Application No. 2012/0171280, which is hereby incorporated by reference. Nanonization may be used to further decrease the size and increase the surface area of the liposomes.

In some embodiments, the present invention utilizes an emulsion vehicle that contains a lignite extract, which is similar to humic or fulvic acid, to act as complexing agents for various active ingredients. This emulsion vehicle improves the bioavailability and the pharmacokinetic profiles of the actives, while decreasing the quantity that needs to be taken for therapeutic effect. Like the liposomal vehicle, the emulsion vehicle can be utilized for mucosal or transdermal delivery and is capable of delivering its payload to the intracellular environment despite any early breakdown of the emulsion vehicle. In an emulsion vehicle, the lignite extract or fluvic acid contained within its aqueous phase acts as a stabilizer for the emulsion, as well as an active ingredient. Common emulsions do not tend to form spontaneously and energy input, including shaking, stirring, homogenizing, or exposure to ultrasound is needed to form an emulsion. It is well known in the art how to create an emulsion vehicle. Nanonization may be used to further decrease the size and increase the surface area of the emulsion vehicles.

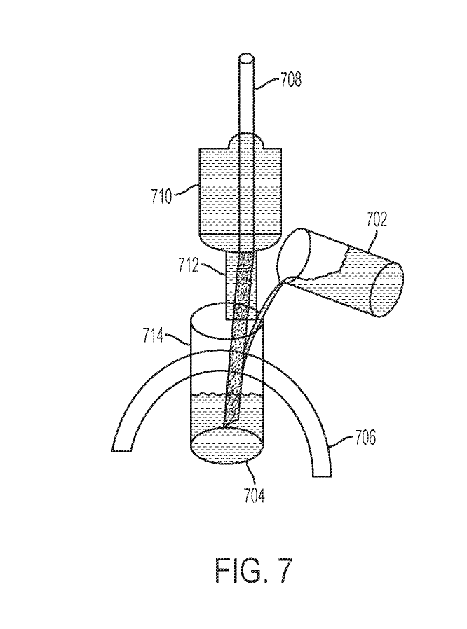

FIG. 7 illustrates a process of creating an emulsion vehicle according to some embodiments of the present invention. A base 706 stabilizes a stand 708 for holding a housing 710 for a motor. The housing 710 is connected to a mount for a probe or mixer 712. The motor (not shown) in the housing 710 causes the probe or mixer 712 to agitate the solution 704 in the container 720. Generally, the solution 704 may be a solvent designed to interact with the aqueous solution 702, which may contain the active ingredients, complexing agents, etc. When the aqueous solution 702 is fully combined with the solvent 704, the probe or mixer 712 agitates the combined solution until it is homogeneous. The combined solution contains the emulsion vehicles to be used in the present invention. The makeup of the emulsion vehicles is determined by the properties of the materials and substances involved, the methods of emulsion, the time and power used in agitation, and the temperature during emulsion.

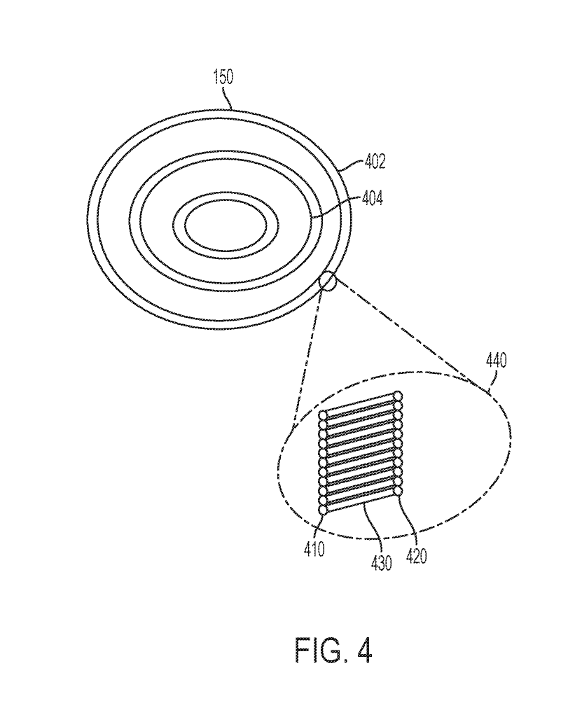

The present invention employs membrane and surface agents to increase absorption levels in both mucosal delivery and transdermal delivery as well as stabilize the liposomal vehicle or emulsion vehicle. FIG. 4 illustrates a cross-section view of a membrane 440 of a liposome. Specifically, a large multimellar vesicle 150 includes a first lipid layer 402 and a second lipid layer 404. The exploded view of the membrane 440 of the first lipid layer 402 shows an outer layer 420 and an inner core bilayer 410. The outer layer 420 and the inner core bilayer 410 are made up of phospholipids. Membrane agents 430 hold the phospholipids together to make up the membrane 440. Surface agents may be combined with the outer layer 420 to interact with target cells or environments. A membrane agent 430 is a molecule that will bind with the membrane of the substance or liposome to improve its ability to enter a target cell. A surface agent is a molecule that will bind with the outer layer of the substance suspended in an emulsion to improve its ability to enter a target cell. In some embodiments, sphingomyelin, cholesterol, ceramides, and cerebrosides may be used as membrane or surface agents to improve the in vitro stability of the vehicles.

These delivery systems allow for compounds such as sildenafil citrate, sildenafil lactate, legacy-patented formulations, pharmaceutical active ingredients, homeopathic agents, traditional Chinese medicinal agents, nutraceuticals, and cosmeceuticals. These substances can be delivered in a variety of dosage forms for oral mucosal tissue through delivery vehicles, such as chewing gum, sublingual tablets, troches, lozenges, strips, and buccal patches. For actives with a limited oral bioavailability due to degradation in the gastrointestinal tract and/or first pass metabolism in the liver, oral transmucosal drug delivery presents a viable option. These substances can also be delivered in a variety of dosage forms for transdermal delivery, including creams, gels, reservoir patches, matrix patches, multi-layer drug-in adhesive patches, and single-layer drug-in adhesive patches. Subcutaneous or intramuscularly deliveries can be made through injectables or implants, and dermal delivery can be made through creams, gels, and serums. Intravenous delivery can be made through an infusion dosage form, and rectal or vaginal delivery can be made through a variety of dosage forms, including suppositories, enemas, catheters, and bulb syringes.

As mentioned above, U.S. Patent Application No. 2016/0331693 is hereby incorporated by reference, including the liposomes and formulations presented therein. For example, sildenafil-analogues, including sildenafil, homosildenafil, hydroxyhomosildenafil, desmethylsildenafil, acetidenafil, vardenafil, and udenafil, may be used as the active ingredient in the present invention. The corresponding sildenafil may react with statin derivatives, γ-polyglutamic acid derivative, vitamin, or sodium CMC to form the monoquaternary amine complex salts of sildenafil-analogues and amine complex salts of udenafil-analogues for delivery to the patient. Thus, the lactone ring, ester and protected derivatives of the statins are available to prepare the above sildenafil-analogues monoquaternary amine complex salts or udenafil-analogues amine complex salts.

Preferred statin derivatives for use with the present invention may include atorvastatin, lovastatin, pitavastatin, rosuvastatin, and simvastatin, γ-polyglutamic acid derivatives may be selected from alginate sodium, the γ-polyglutamic acid, the sodium polyglutamate, and the GLT is referred as the co-polymer of lysine, glutamate, and tyrosine, and the calcium polyglutamate-alginate sodium, vitamin is selected from retinoic acid, ascorbic acid, folic acid, gammalinolenic acid, nicotinic acid, and pantothenic acid. Thereby, the sildenafils-γ-polyglutamic acid, sildenafils-lovastatinic acid, sildenafils-simvastatinic acid, sildenafils-pramastatinic acid, sildenafils-pitavastatin, sildenafils-rosuvastatin, sildenafil-L-arginine, sildenafil-CMC, sildenafil-mevastatinic acid, sildenafil-rosuvastatinic acid, sildenafils-lovastatinic acid, udenafil-CMC, udenafil-nicotinic acid, and udenafil-L-retinoic acid may be obtained from the sildenafil/statin combinations.

Further, the present invention may include delivery of the following peptides, including but not limited to, insulin and derivatives of insulin, such as lispro. C-peptide, glucagon-like peptide 1 (GLP 1) and all active fragments, human amylin and synthetic forms of amylin, such as pramlintide, parathyroid hormone (PTH) and active fragments thereof (e.g., PTH1-34), calcitonin, human growth hormone (HGH), erythropoietin (EPO), macrophage-colony stimulating factor (M-CSF), granulocyte-macrophage-colony stimulating factor (GM-CSF), and interleukins may also be delivered. Smaller molecules may include nitroglycerin, sumatriptan, narcotics (e.g., fentanyl, codeine, propoxyphene, hydrocodone, and oxycodone), benzodiazepines (e.g., alprazolam, clobazam, clonazepam, diazepam, flunitrazepam, lorazepam, nitrazepam, oxazepam, temazepam, and triazolam), phenothiazines (chlorpromazine, fluphenazine, mesoridazine, methotrimeprazine, periciazine, perphenazine, prochlorperazine, thioproperazine, thioridazine, and trifluoperazine), and selective serotonin reuptake inhibitors (SSRIs) (e.g., sertraline, fluvoxamine, fluoxetine, citalopram, and paroxetine). The liposome and emulsion delivery vehicles may also deliver larger molecules, cells, and stem cells to the target.

In some embodiments, sildenafil citrate (20 mg), atorvastatin (4 mg, 8 mg, 12 mg), osimertinib (40 mg, 80 mg), afatinib dimaleate (20 mg, 30 mg, 40 mg), and digoxin (0.05 mg, 0.10 mg) may be delivered sublingually. Metformin (500 mg/5 mL, 100 mg/5 mL) may be delivered as a creme. Amoxicillin (250 mg, 500 mg), amoxicillin and potassium clavulanate (250 mg/62.5 mg, 500 mg/125 mg), azithromycin (165 mg, 330 mg), clarithromycin (250 mg, 500 mg), erythromycin (250 mg, 500 mg), testosterone/anastrozole (6 mg/0.2 mg), zidovudine (400 mg, 500 mg, 600 mg), oseltamivir (40 mg, 60 mg, 85 mg), fentanyl citrate (25 mcg/mL, 50 mcg/mL), and chloroquine phosphate (400 mg, 900 mg) may be delivered as a patch. Cisplatin (0.3 mg/mL) and nivolumab (3 mg/mL) may be delivered through IVs. Lipoplatin may also be delivered to the patient through the methods disclosed herein. U.S. Patent Application No. 2014/0271821, which is hereby incorporated by reference, discloses numerous liposomal cisplatin compositions and methods for preparing liposomal cisplatin, which may be delivered to the patient through the methods disclosed herein.

The present invention has superior intercellular delivery because of the lignite extract (or humic or fluvic acid) complexing agents that are combined within the liposomal delivery vehicle. A complexing agent is a molecule or substance that will bind with an active ingredient to allow the substance to efficiently absorb at its target. This improved liposome is capable of delivering its payload to the intercellular environment despite any early breakdown of the liposomal vehicle before it reaches its target cell. Specifically, even if the outer membrane of the liposome breaks down, the lignite extract can accompany the active ingredient into the cell independent of the liposomal vehicle. This complexing agent (lignite extract) acts like a cage for at least a portion of the active ingredient that delivers the active ingredient to its target.

The use of lignite extract solves the problem of poor delivery into the cerebral spinal fluid. The liposomal vehicle of the present invention is able to directly traverse through the length of any cranial nerve in the intranasal cavity to the cerebral spinal fluid. In traditional liposomal vehicles, the actives would have to bind to the extracellular receptors on the cranial nerve to be taken up by it, but here the liposome can traverse the cranial nerve. Further, many active ingredients can irritate the tissues of the central nervous system, however, by encapsulating the active ingredients in the liposome and binding the active ingredients to the lignite extract, the present invention prevents the active ingredients from being able to interact with extracellular receptors on central nervous system tissue, endothelium tissue, vaginal tissue, or rectal tissue as the liposomal vehicle is traveling to its intracellular target.

The use of lignite extract (or humic acid or fulvic acid) also improves the stability of potentially many different actives because the lignite/active complex is protected from hydrolosis. More specifically, the lignite extract helps to prevent the active ingredient from being broken down when it interacts with water.

In some embodiments, the liposomal vehicle contains a specific mixture of naturally occurring phospholipids (i.e., phosphatidic acid, phosphatidyl-choline, phosphatidyl-glycerol, phosphatidyl-inositol, phosphatidyl-serine, digalactosyldiacylglyceride, and monogalactosyldiacylglyceride), which make up the lipid outer shell and allow for increased stability of the inner core bilayer (See FIG. 4). These phospholipids (1) enable the liposome to be ultradeformable, which allow penetration through the narrow pores of the skin without measurable loss, and (2) protect the encapsulated active ingredient from enzymatic and metabolic degradation. Elastic and flexible membranes improve penetration of the liposome into the skin. Unlike synthetic phospholipids, which are used in many traditional liposomes, naturally occurring phospholipids better mimic the body's intrinsic mixture of phospholipids.

These naturally occurring phospholipids further assist in getting the active ingredients to the nerves of the dermis and subcutaneous tissue more easily, which occurs because of their affinity to the trpv-1 receptor of nerves.

The liposomal vehicle may contain sphingomyelin and cholesterol in the liposome's membrane, which improve the in vitro stability of the liposomal vehicle as it passes through the mucosal tissue. Ceramide and cerebroside may be used in the liposome's membrane to improve the in vitro stability of the liposomal vehicle as it passes through dermal tissue. Due to the increased in vitro stability, the bioavailability of the active ingredient is further increased. These features of the liposomal vehicle may be used to help deliver active ingredients rectally, vaginally, otically, intraosseously, intraperitoneally, ophthalmically, subcutaneously, intravenously, intrathecally, intramuscularly, intranasally, and directly to organs and tissues through invasive surgery. Specifically, a scaffold system may be utilized during invasive surgery to deliver active ingredients or cells needed to be delivered directly to target tissue. In this embodiment, the liposomal vehicle may be mixed with allogenic extracellular amniotic tissue.

The present invention utilizes a number of different components to improve bioavailability. One such component, is the positively charged liposomal vehicle that adheres to the oral mucosal tissue due to its negatively charged membrane. Specifically, the membrane of the liposome is positively charged due to the presence of trimethlglycine. The ratio of phospholipids also helps with the adhesive properties. There are numerous advantages of this positively charged liposomal vehicle, including (1) more of the active ingredient will be delivered to the mucosal tissue and less will be swallowed by the user, (2) faster onset of action, and (3) improved bioavailability of the active ingredient.

Dosages and concentrations of the active ingredients depend upon their bioavailability, delivery method, and the condition to be treated. The compositions may also contain one or more solubilizing agents, diluents, binders, lubricants, or stabilizers. These compounds or molecules assist with delivery of the active ingredients in different ways. Solubilizing agents may be included with the active ingredient to promote rapid dissolution in aqueous media. These may include wetting agents (polysorbates and poloxamers), non-ionic, and ionic surfactants, food acids and bases (sodium bicarbonate), and alcohols and buffer salts for pH control. Diluents or fillers may be used to fill the bulk of the delivery vehicle to a practical size. For example, fillers may be added to make a tablet of a desired size and shape. Suitable fillers include dicalcium phosphate dihydrate, calcium sulfate, lactose, sucrose, mannitol, sorbitol, cellulose, microcrystalline cellulose, powdered cellulose, kaolin, sodium chloride, dry starch, hydrolyzed starches, sugar, dextrates, dextrin, potassium chloride, talc, and many others. Binders may be added to impart cohesive qualities to a solid dosage formulation and ensure that a tablet remains intact. Suitable binders include starch, pregelantinized starch, gelatin, sugars, dextrin, maltodextrin, zein, polyethylene glycol, waxes, natural and synthetic gums, celluloses, hydrogenated vegetable oil, synthetic polymers, and many others.

Lubricants may be used to assist with tablet manufacture and may include magnesium stearate, calcium stearate, stearic acid, glyceryl behenate, glyceral monostearate, glyceryl palmitostearate, hydrogenated castor oil, hydrogenated vegetable oil, and many others. Stabilizers, which are used to delay drug decomposition reactions, such as oxidative reactions, may also be used. Surfactants or surface agents may be anionic, cationic, amphoteric, or nonionic surface agents. Suitable anionic surfactants include those containing carboxylate, sulfonate, and sulfate ions, while suitable anionic surfactants include sodium, potassium, ammonium of long chain alkyl sulfonates, alkyl sulfonates, dialkyl sodium sulfosuccinates, diakyl sodium sulfosuccinates, and alkyl sulfates.

As mentioned above, numerous formulas, dosages and concentrations may be used for the present invention. Below is an ingredient list for an exemplary embodiment that may be used for most, if not all, of the active ingredients and delivery methods disclosed herein. This ingredient list may be used for a liposomal or an emulsion delivery vehicle. Further, the complexing agents or phospholipids may be used together or only one or more may be used in the liposomal or emulsion delivery vehicle. For example, (1) lignite extract, humic acid, and fulvic acid may all be used as complexing agents, (2) lignite extract may be used only with humic acid, or (3) lignite extract may be the only complexing agent used.

| Ingredient List | Use |
|---|---|
| Lignite Extract | Complexing Agent, Complexes with |
| | Active Ingredient |
| Humic Acid | Complexing Agent, Complexes with |
| | Active Ingredient |
| Fulvic Acid | Complexing Agent, Complexes with |
| | Active Ingredient |
| Phosphatidyl- | A component of the encapsulating |
| choline | vesicle of the active ingredient |
| Phosphatidyl- | A component of the encapsulating |
| glycerol | vesicle of the active ingredient |
| Phosphatidyl- | A component of the encapsulating |
| inositol | vesicle of the active ingredient |
| Phosphatidyl- | A component of the encapsulating |
| serine | vesicle of the active ingredient |
| Digalactosyl- | A component of the encapsulating |
| diacylglyceride | vesicle of the active ingredient |
| Monogalactosyl- | A component of the encapsulating |
| diacylglyceride | vesicle of the active ingredient |
| Sphingomyelin | Vesicle Stabilizing Agent |
| Cholesterol | Vesicle Stabilizing Agent |
| Ceramide | Vesicle Stabilizing Agent |
| Cerebroside | Vesicle Stabilizing Agent |
| Natural Mint | Flavoring |
| Flavor | |
| Stevia | Zero Calorie sweetener, effective |
| | in masking bitterness |
| Magnesium | Lubricant to prevent tablet and capsule |
| Stearate | content from sticking to the |
| | machinery that processes them |
| Sodium | Provides Foaming Action |
| Bicarbonate | |
| Microcrystalline | Bulking agent, disintegrant, binder, and |
| cellulose 105 | lubricant. Also acts as a stability enhancer. |

The use of lignite extract in the present invention may also assist with the issue of poor taste or bitterness of active ingredients. In humans, bitter taste perception is mediated by 25 different g-proteins coupled receptors (GPCRs) of the hTASwR family. Due to the fact that the majority of the desired active ingredient will be encapsulated in a liposomal vehicle, much less of the active ingredient will be able to interact with the 25 GPCRs that cause a perception of bitter taste. The GPCRs are also protected from the desired actives because most actives that are not located within the liposome are complexed with lignite extract or humic acid or fulvic acid, and the complex (active ingredient—lignite extract/humic acid/fulvic acid) may not be able to interact in the same way with the GPCRs. The uncomplexed active ingredients may not cause the same degree of bitter taste perception due to the fact that they are bound with the lignite extract.

The use of lignite extract also enables the present invention to deliver higher molecular weight substances to the target cellular environments, including both active ingredients and other types of cells. As mentioned above, the lignite extract provides support to the liposomal vehicle by making it ultradeformable and making it capable of penetrating the squamous epithelium and getting into systemic circulation. It does this by decreasing the liposomal membrane fluidity in the surface region, while also stiffening the central part of the liposomal bilayer. The lignite extract electrostatically stabilizes the central part of the liposomal bilayer by making the inside of the bilayer negatively charged and making the outside layer of the liposome positively charged, which further stabilizes the central party of the bilayer.

In some embodiments, the liposomal delivery system can be used in conjunction with a scaffold system during invasive surgery to deliver active ingredients or cells directly to targeted tissue. The protections and ultradeformability of the liposomal vehicle allows for more of the active ingredient, therapeutic cell, or stem cell product to reach the targeted organ or tissue.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A liposomal delivery vehicle for delivering an active ingredient to a target cellular environment comprising:
at least one complexing agent that interacts with said active ingredient, wherein said at least one complexing agent comprises a lignite extract;
an encapsulating vesicle for enclosing said active ingredient, wherein said encapsulating vesicle comprises at least one phospholipid to create a membrane around said active ingredient; and
at least one membrane agent that binds to said membrane of said liposomal delivery vehicle;
wherein said interaction between said lignite extract and said active ingredient enables said active ingredient to be delivered to said target cellular environment even if said encapsulating vehicle degrades before reaching said target cellular environment.

2. The liposomal delivery vehicle of claim 1 wherein said at least one complexing agent further comprises humic acid.

3. The liposomal delivery vehicle of claim 2 wherein said at least one complexing agent further comprises fulvic acid.

4. The liposomal delivery vehicle of claim 1 wherein said at least one membrane agent comprises sphingomyelin.

5. The liposomal delivery vehicle of claim 1 wherein said at least one membrane agent comprises at least one ceramide molecule.

6. The liposomal delivery vehicle of claim 1 wherein said at least one membrane agent comprises at least one cerebroside molecule.

7. The liposomal delivery vehicle of claim 1 wherein said active ingredient is a sildenafil-analogue molecule and said lignite extract interacts with said sildenafil-analogue to improve a bioavailability of said liposomal delivery vehicle.

8. The liposomal delivery vehicle of claim 7 wherein said active ingredient further comprises at least one statin derivative and said lignite extract further interacts with said statin derivative.

9. A liposome for delivering an active ingredient to a target cellular environment comprising:
at least one complexing agent that interacts with said active ingredient, wherein said at least one complexing agent comprises a lignite extract; and
at least one membrane agent that binds to a membrane of said liposome;
wherein said active ingredient is enclosed within said membrane of said liposome and wherein said interaction between said lignite extract and said active ingredient enables said active ingredient to be delivered to said target cellular environment even if said membrane degrades before reaching said target cellular environment.

10. The liposome of claim 9 wherein said at least one complexing agent further comprises humic acid.

11. The liposome of claim 9 wherein said at least one complexing agent further comprises fulvic acid.

12. The liposome of claim 9 wherein said at least one membrane agent comprises sphingomyelin.

13. The liposome of claim 9 wherein said active ingredient is a sildenafil-analogue molecule and said lignite extract interacts with said sildenafil-analogue to improve a bioavailability of said liposome.

14. The liposome of claim 13 wherein said active ingredient further comprises at least one statin derivative and said lignite extract further interacts with said statin derivative.

15. An emulsion delivery vehicle for delivering an active ingredient to a target cellular environment comprising:
at least one complexing agent that interacts with said active ingredient, wherein said at least one complexing agent comprises a lignite extract; and
an encapsulating vesicle for enclosing said active ingredient;
wherein said interaction between said lignite extract and said active ingredient enables said active ingredient to be delivered to said target cellular environment even if said encapsulating vehicle degrades before reaching said target cellular environment.

16. The emulsion delivery vehicle of claim 15 wherein said at least one complexing agent further comprises humic acid.

17. The emulsion delivery vehicle of claim 15 wherein said at least one complexing agent further comprises fulvic acid.

18. The emulsion delivery vehicle of claim 17 wherein said at least one complexing agent further comprises humic acid.

19. The emulsion delivery vehicle of claim 15 wherein said active ingredient is a sildenafil-analogue molecule and said lignite extract interacts with said sildenafil-analogue to improve a bioavailability of said emulsion delivery vehicle.

20. The emulsion delivery vehicle of claim 19 wherein said active ingredient further comprises at least one statin derivative and said lignite extract further interacts with said statin derivative.
